(19) United States
(12) Patent Application Publication
(10) Pub. No.: US 2015/0099928 A1
(43) Pub. Date: Apr. 9, 2015

(54) INJECTABLE COMPOSITIONS

(71) Applicant: Boston Scientific Scimed, Inc., Maple Grove, MN (US)

(72) Inventors: Paul Smith, Smithfield, RI (US); Felipe Torres, Chicago, IL (US); Samuel Raybin, Marlborough, MA (US); Mary Jo Timm, Littleton, MA (US)

(73) Assignee: Boston Scientific Scimed, Inc., Maple Grove, MN (US)

(21) Appl. No.: 14/508,347

(22) Filed: Oct. 7, 2014

Related U.S. Application Data
(60) Provisional application No. 61/887,727, filed on Oct. 7, 2013.

(51) Int. Cl. A61K 47/38; A61B 17/22; A61M 5/178; A61B 17/34; A61K 47/36; A61B 1/018

(52) U.S. Cl. CPC A61K 47/38; A61K 47/36; A61B 1/018; A61M 5/178; A61B 17/3478; A61B 17/22; A61B 2017/0034
USPC 600/104; 514/777; 514/781

(57) ABSTRACT

In one aspect, the invention provides injectable compositions that comprise a suitable hydrophilic polymer (e.g., a suitable polysaccharide) and water, as well as other optional components. In various embodiments, the composition may be provided in a suitable container, for example, in a pre-loaded syringe. In another aspect, methods of performing medical procedures in a tract of a body are provided. In yet another aspect, the invention provides systems for performing medical procedures in a tract of a body.

### STATEMENT OF RELATED APPLICATION

This application claims the benefit of U.S. Provisional Application Ser. No. 61/887,727, filed Oct. 7, 2013 and entitled “Injectable Compositions,” the disclosure of which is hereby incorporated by reference in its entirety.

### FIELD OF THE INVENTION

This invention relates to medical articles and related methods thereof. In particular embodiments, this invention relates to articles and related methods for performing agent-assisted endoscopic procedures in, for example, a gastrointestinal (GI) tract of a patient.

### BACKGROUND

An endoscope is a medical device that enables viewing of the interior of a body cavity or hollow organ without employing invasive surgical procedures. The endoscope includes a flexible elongated body (e.g., a tube) having a suitable imaging device at its distal end portion. The endoscope may be inserted through a naturally occurring opening, such as the esophagus or rectum, or through a small incision surgically made in the body. Suitable surgical instruments may be passed through the endoscope to perform various medical procedures, such as, for example, tissue sampling or removal of diseased tissue or polyps.

Endoscopic procedures are commonly used for diagnosis and/or treatment of the GI tract. For example, an endoscopic procedure may be performed to take tissue samples from the GI tract for pathological evaluation and/or therapeutic purposes. For instance, with advances in the imaging technology, endoscopic procedures may be used to accurately detect and remove pre-cancerous mucosal tissue or tumors from various locations in the GI tract.

Interventional endoscopists perform various tasks including fluid-assisted polypectomy, endoscopic mucosal resection (EMR), and endoscopic submucosal dissection (ESD) procedures to remove pre-cancerous or cancerous mucosal tissue from the GI tract. Such fluid-assisted procedures may involve injecting a fluid cushion into submucosal tissue (e.g., cushioning) or injecting a fluid between target tissue layers (e.g., dissection) so as to raise or separate the target tissue layer in order to safely perform the procedure (e.g., by preventing or reducing risks of perforating the GI tract).

Injectable compositions, however, dissipate and therefore may not raise or separate the target tissue layer for the entire duration of the procedure. If the fluid is dissipated, the endoscopist must re-inject the fluid to assure the target tissue layer remains raised or bulked. The more times the tissue is pierced with an injection needle to inject the fluid, the more holes that are created for the fluid to leak out.

### SUMMARY OF THE INVENTION

According to one aspect, the invention provides injectable compositions suitable for performing medical procedures. In various embodiments, the injectable compositions (also referred to as injectable fluids) may comprise a suitable hydrophilic polymer (e.g., a suitable polysaccharide) and water, as well as other optional components. In various embodiments, the composition may be provided in a suitable container, for example, in a pre-loaded syringe.

In another aspect, a method of performing a medical procedure in a tract of a body is provided. The method may include injecting an injectable material proximate a target site between a first tissue layer and a second tissue layer. In various embodiments, the injectable compositions may be injected using an ordinary syringe. In some embodiments, the medical procedures may include removing tissue from the target site.

In another aspect, the invention may provide a system for performing a medical procedure. The system may include (a) a syringe filled with an injectable composition (e.g., an injectable composition as described herein) and (b) an elongated hollow member having a proximal end configured to engage the syringe in order to receive an injectable material from the syringe and a sharp distal end configured to pierce tissue and deliver the injectable material.

These and other aspects, embodiments and advantages of the present invention will become immediately apparent to those of ordinary skill in the art upon review of the Detailed Description and claims to follow.

### BRIEF DESCRIPTION OF THE DRAWINGS

FIG. 1 is a schematic cross-sectional view of mucosal and submucosal tissue layers in a GI tract, showing diseased tissue in the mucosal tissue layer.

FIG. 2 is a schematic cross-sectional view of mucosal and submucosal tissue layers in the GI tract, illustrating an exemplary method step of injecting an injectable composition between the mucosal and submucosal tissue layers.

FIG. 3 is a schematic cross-sectional view of mucosal and submucosal tissue layers in the GI tract, showing the raised mucosal tissue layer resulting from the injection of the injectable material.

FIG. 4 is a schematic cross-sectional view of mucosal and submucosal tissue layers in the GI tract, illustrating an exemplary method step of removing diseased tissue from the mucosal tissue layer.

FIG. 5 is a schematic cross-sectional view of mucosal and submucosal tissue layers in the GI tract, showing the condition of the mucosal and submucosal tissue layers after the endoscopic procedures.

FIG. 6 is a schematic cross-sectional view of mucosa, submucosa and muscularis propria tissue in a GI tract, showing diseased tissue in the mucosal and submucosal layers.

FIG. 7 is a schematic partial cross-sectional view of mucosa, submucosa and muscularis propria tissue in a GI tract, illustrating an exemplary method step of injecting an injectable composition into the submucosa.

FIG. 8 is a schematic partial cross-sectional view illustrating an exemplary method step of injecting an injectable composition in conjunction with a peroral endoscopic myotomy (POEM) procedure.

### DESCRIPTION OF THE EMBODIMENTS

A more complete understanding of the present invention is available by reference to the following detailed description of numerous aspects and embodiments of the invention. The detailed description of the invention which follows is intended to illustrate but not limit the invention.

According to one aspect, the present disclosure provides injectable compositions that are suitable for performing medical procedures. In various embodiments, the compositions are sterile, for example, having been sterilized by heat, radiation or sterile filtration. In various embodiments, the compositions may comprise a suitable hydrophilic polymer and water, as well as other optional agents.

Preferred hydrophilic polymers for use in the present disclosure include polysaccharides. Polysaccharides for use in conjunction with the present disclosure include linear polysaccharides such as cellulose, amylose, pectin, alginates, and derivatives of the forgoing, including alkyl cellulose polymers such as methyl cellulose (MC), hydroxyalkyl celluloses such as hydroxypropyl cellulose (HPC) and hydroxypropylmethyl cellulose (HPMC), and carboxyalkyl celluloses and their salts including carboxymethyl celluloses (CMC). Counterions for use in carboxyalkyl celluloses include Group I cations such as sodium and potassium, Group II cations such as magnesium and calcium, and mixtures of the foregoing. Carboxymethyl cellulose is manufactured with molecular weights ranging, for example, from 50,000 to 800,000 Da. Sodium CMC is preferred in certain embodiments.

Polysaccharides for use in conjunction with the present disclosure also include glycosaminoglycans, preferably, non-sulfated glycosaminoglycans such as hyaluronic acid and its salts, desulfated heparin, desulfated chondroitin sulfate and desulfated dermatan sulfate. Hyaluronic acid and its salts (also called hyaluronan, hyaluronate or HA) are anionic, nonsulfated glycosaminoglycans. HA is distributed widely throughout connective, epithelial, and neural tissues. Counterions for use in hyaluronic acid salts include Group I cations such as sodium and potassium, Group II cations such as magnesium and calcium, and mixtures of the foregoing. HA can range, for example, from 5,000 to 20,000,000 Da in vivo. Hyaluronic acid sodium salt is preferred in certain embodiments.

Polysaccharides for use in conjunction with the present disclosure also include polysaccharides comprising a main chain and a plurality of monosaccharide side groups. Examples of such compounds include galactomannans which are polysaccharides having a mannose backbone with galactose side groups (e.g., a (1-4)-linked beta-D-mannopyranose backbone with branchpoints from their 6-positions linked to alpha-D-galactose, i.e., 1-6-linked alpha-D-galactopyranose), such as guar gum, fenugreek gum, tara gum, locust bean gum and carob gum. Polysaccharides for use in conjunction with the present disclosure also include polysaccharides comprising a main chain and a plurality of oligosaccharide side groups (where “oligosaccharide” is defined herein as polysaccharide chains of 2, 3, 4, 5, 6, 7, 8, 9 or 10 saccharide groups), including xanthan gum.

Polysaccharides further include branch-on-branch polysaccharides such as amylopectin, gum arabic, arabinoxylan, among others.

Polysaccharides for use in conjunction with the present disclosure may vary widely in molecular weight, ranging, for example, from 5 kDa or less to 20,000 kDa or more, for example, ranging from 5 kDa to 10 kDa to 25 kDa to 50 kDa to 75 kDa to 100 kDa to 250 kDa to 500 kDa to 750 kDa to 1000 kDa to 2500 kDa to 5000 kDa to 7500 kDa to 10,000 kDa to 15,000 kDa to 20,000 kDa (i.e., ranging between any two of the preceding numerical values).

Polysaccharides concentrations in the injectable compositions of the present disclosure may vary widely, ranging, for example, 0.05% w/w or less to 0.5% w/w or more, for instance ranging from 0.05% w/w to 0.075% w/w to 0.1% w/w to 0.125% w/w to 0.15% w/w to 0.175% w/w to 0.2% w/w to 0.225% w/w to 0.25% w/w to 0.275% w/w to 0.3% w/w to 0.325% w/w to 0.35% w/w to 0.375% w/w to 0.4% w/w to 0.425% w/w to 0.45% w/w to 0.475% w/w to 0.5% w/w.

Addition of polysaccharides to water results in an increase in viscosity. In various embodiments, the viscosity of the injectable compositions ranges from 100 cps to 5000 cps (e.g., ranging from 100 cps to 200 cps to 500 cps to 1000 cps to 2000 cps to 5000 cps). Solution viscosity is a function of both the polymer concentration and the molecular weight of the polymer. At a given constant weight concentration, solution viscosity exhibits an exponential relationship with the molecular weight of the polymer used to adjust the viscosity of the solution. Consequently, an increase in molecular weight for a given polymer will allow a lower concentration (by weight) of the polymer to be used to achieve a given viscosity.

In some embodiments, the injectable compositions for use in conjunction with the present disclosure include non-Newtonian fluids that exhibit decreasing viscosities under shear, including pseudoplastic fluids and thixotropic fluids. Thixotropic fluids exhibit this change as a result of time under constant shear while pseudoplastic fluids exhibit this change as a result of increasing the rate of shear stress. Examples of thixotropic fluids include solutions of gums such as xanthan gum or guar gum. Examples of pseudoplastic solutions include solutions containing hyaluronic acid and salts thereof and celluloses such as alkyl celluloses, hydroxy alkyl celluloses and carboxyalkyl celluloses, among polymers.

In some embodiments the injectable compositions for use in conjunction with the present disclosure are colloids. As defined herein a colloid is a system that has a continuous liquid phase in which large molecules or small solid particles (e.g., particles ranging from 1 to 1,000 nm in diameter) are suspended. In various embodiments, the injectable compositions are hydrocolloids (i.e., a colloid system wherein the colloid particles are hydrophilic polymers dispersed in water).

In some embodiments, the injectable compositions further comprise one or more optional agents. Examples of optional agents include imaging agents, such as, for example, colorants and dyes (e.g., indigo carmine or methylene blue), fillers, and/or therapeutic agents such as cancer-treating agents (e.g., endostatin, etc.), hormones, anti-inflammatory agents, antibiotics, pain-relieving agents, antimicrobial agents (e.g., antibacterial agents, anti-fungal agents, etc.) as well as combinations of the same.

In other embodiments, the injectable compositions consist essentially of polysaccharide and water.

It has been noted by the present inventors that solutions with higher polymer concentrations can cause bruising to take place at the injection site. Without wishing to be bound by theory, it is believed that the injection of a hydrocolloid with sodium ions, such as sodium carboxy methyl cellulose (SCMC), sodium hyaluronate (SH) and sodium alginate (SA), with tissue under tension causes bruising of tissue layers, and it is further theorized that the mechanism for such bruising is the imbalance of ions, including sodium ions, between the injectable composition and the tissue.

In certain embodiments, the injectable compositions for use in conjunction with the present disclosure contain sodium at a concentration of 150 mmol/l or less, for example, ranging from 150 mmol/l to 145 mmol/l to 140 mmol/l to 135 to mmol/l to 130 mmol/l to 125 mmol/l to 120 mmol/1 to 115 mmol/l to 110 mmol/l to 105 mmol/l to 100 mmol/l or less. In certain embodiments the sodium concentration ranges from 100 mmol/l to 120 mmol/l.

In certain embodiments, the injectable compositions of the present disclosure have an osmolarity of less than 310 milliosmoles/liter (mOsm/L), for example, for example, ranging from 310 mOsm/L to 300 mOsm/L to 290 mOsm/L to 280 mOsm/L to 270 mOsm/L to 250 mOsm/L to 225 mOsm/L to 250 mOsm/L, or less.

Charged polymers induce osmotic pressure not only because of the molecules themselves, but also due to counterions associated with the polymers. As noted above, an increase in molecular weight for a given polymer will allow a lower concentration (by weight) of the polymer to be used to achieve a given viscosity. A lower weight concentration of polymer will result in a lower osmotic pressure for the composition (e.g., due to the lower concentration of the polymer itself, and where the polymer is ionic, a lower concentration of counterions, such as sodium counterions). In this way, viscosity and osmotic pressure can be varied by varying concentration and molecular weight. For example, injectable compositions may be formed with similar osmotic pressure properties (e.g., osmolarity) but with varying viscosities, and vice versa.

In certain embodiments, the injectable compositions of the present disclosure may comprise a suitable buffer in an amount sufficient to achieve an appropriate in vivo pH at the target site. Examples of suitable buffers include phosphate buffered saline (PBS), Tris (i.e., tris(hydroxymethyl)aminomethane) buffer, Tris-buffered saline, HEPES (i.e., 4-(2-hydroxyethyl)-1-piperazineethanesulfonic acid) buffer, and HEPES-buffered saline, among many others. In other embodiments, a buffer is not included in the injectable compositions of the present disclosure.

In certain embodiments, the injectable compositions of the present disclosure are supplied in one or more syringes. Such syringes may include a barrel having an opening to receive a plunger at its proximal end and having and a fitting (e.g., a luer fitting or another suitable fitting) at its distal tip for direct or indirect engagement with an injection needle such that the interior of the syringe barrel is placed in fluid communication with the interior of an injection needle. The barrel may also be provided with a flange at its proximal end for ease of engagement and a scale for determining the volume of fluid remaining in the barrel. Suitable syringe volume may range, for example, from 5 cc or less to 50 cc or more, for example, ranging from 5 cc to 7.5 cc to 10 cc to 12.5 cc to 15 cc to 20 cc to 25 cc to 30 cc to 40 cc to 50 cc, preferably from 7.5 cc to 12.5 cc.

A suitable injection needle may be provided, for example, an endoscopic injection needle that comprises a flexible tubular portion (catheter portion) having a hollow needle tip at its distal end and a suitable fitting/adaptor (e.g., a luer fitting) for engagement with a syringe barrel at its proximal end. Suitable needle gauge may vary from 20 gauge or less to 27 gauge or more, for example, from 20 gauge to 21 gauge to 22 gauge to 23 gauge to 24 gauge to 25 gauge to 26 gauge to 27 gauge, preferably 23 gauge to 25 gauge. Suitable endoscopic injection needle length may range, for example, from 200 cm to 240 cm.

In certain aspects, the present disclosure pertains to surgical procedures which employ the injectable compositions described herein. While certain embodiments of the disclosure are described herein in connection with particular endoscopic procedures in the GI tract, for instance, endoscopic mucosal resection (EMR), endoscopic submucosal dissection (ESD) and peroral endoscopic myotomy (POEM), embodiments of the disclosure may be used with other suitable endoscopic procedures, or for procedures other than the endoscopic procedures, such as urologic procedures, plastic surgeries, or open invasive surgeries. In addition, embodiments of the disclosure may be applied to numerous parts of a body, other than the GI tract.

EMR is an endoscopic technique developed for removal of sessile or flat neoplasms confined to the superficial layers (mucosa and submucosa) of the GI tract. EMR is typically used for removal of lesions smaller than 2 cm or piecemeal removal of larger lesions. Before the start of an EMR technique, it may be helpful to mark the margins of a targeted lesion with superficial cautery marks. The procedure starts with injection of an injectable composition into the submucosal space under the lesion, creating a “safety cushion.” The cushion lifts the lesion to facilitate its removal and minimizes mechanical or electrocautery damage to the deep layers of the GI tract wall. An “inject-and-cut” technique uses submucosal injection to lift the target lesion and an electrocautery snare to remove the lesion. An “inject-lift-and-cut” technique uses submucosal injection to lift the target lesion and grasping forceps to lift the lesion and an electrocautery snare to remove the lesion. Cap-assisted EMR also uses submucosal injection to lift the target lesion after which the mucosa is suction-retracted into the cap and the lesion removed with an electrocautery snare.

ESD is typically used for en bloc removal of large (usually more than 2 cm), flat GI tract lesions. The procedure is usually done in several steps. First, the margins of the lesion may be marked by electrocautery, and submucosal injection is used to lift the lesion. Then, a circumferential incision into the submucosa is performed around the lesion with specialized endoscopic electrocautery knives. The lesion is then dissected from underlying deep layers of the GI tract wall with the electrocautery knife and removed. Various cutting devices and accessories have been developed specifically for ESD.

Several options are available for collection of resected tissue. For example, after the cap-assisted EMR, the resected pieces can be collected into the cap and retrieved from the patient. As another example, the tissue resected during EMR or ESD can also be collected by specially designed retrieval devices (e.g., nets, baskets, etc.).

In a typical POEM procedure, an initial incision is made in the internal lining of the esophagus. This permits entry of the endoscope to within the wall of the esophagus, where the muscle is exposed. The inner layer of the muscle near the lower esophageal sphincter is then cut (termed myotomy). At the conclusion of the procedure, the esophageal incision is closed (e.g., with endoscopic clips or other suitable closure devices).

Reference will now be made in detail to the exemplary embodiments of the disclosure, examples of which are illustrated in the accompanying drawings. Wherever possible, the same reference numbers will be used throughout the drawings to refer to the same or like parts.

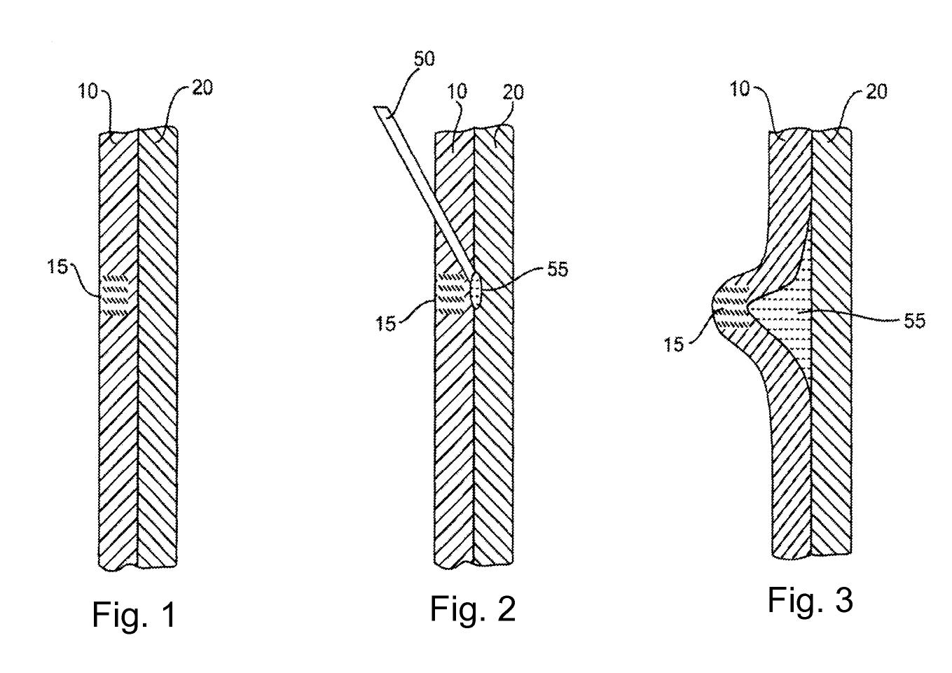

With reference to FIGS. 1-8, the methods and systems according to various exemplary embodiments of the disclosure will be described. As mentioned above, while an embodiment of the disclosure will be described in connection with a particular endoscopic procedure in the GI tract, embodiments of the disclosure may be used with other suitable endoscopic procedures, or for procedures other than the endoscopic procedures.

FIGS. 1-5 are schematic cross-sectional views of a portion in the GI tract showing the mucosal and submucosal tissue layers 10, 20, and illustrate an injection device method for performing a fluid-assisted endoscopic mucosal resection of diseased tissue 15 in the mucosal tissue layer 10 (see FIG. 1), in accordance with one embodiment of the disclosure.

As shown in FIG. 2, an injection device may include an injection needle 50 that may be inserted into rectum or esophagus by any suitable means, such as through a lumen of an endoscope (not shown), so that the distal end portion of the injection needle may be positioned in the vicinity of a target site. The injection needle 50 may include a hollow lumen through which the injectable material 55 may flow. The distal end of the needle 50 may include a sharp edge configured to pierce tissue, so that the distal end of the needle 50 may be positioned between the mucosal tissue layer 10 and the submucosal tissue layer 20 in order to deliver the injectable material 55 at this location. The amount of injectable material to be injected may depend on various factors, such as, for example, type of procedure performed, type of resection instrument used, size of the diseased tissue, or desired degree of lifting.

Upon injection of the injectable material 55, the mucosal tissue layer 10 is dissected from submucosal tissue layer 20 and a portion of the mucosal tissue layer 10 containing the diseased tissue 15 is raised, as shown in FIG. 3.

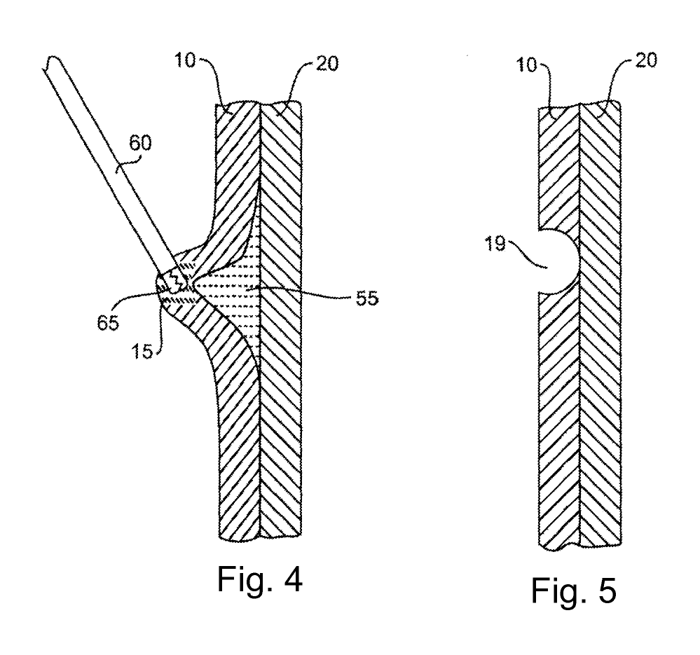

A suitable endoscopic resection device 60 having a suitable cutting member 65 (e.g., snare, knife, biopsy forceps, scissors, etc.) may be used to remove the diseased tissue 15 from the mucosal tissue layer 10, as shown in FIG. 4. The device 60 may be delivered to the tissue site by any suitable means known in the art, such as through a lumen of an endoscope. The injectable material 55 may maintain its stable three-dimensional shape throughout the procedure.

FIG. 5 shows the condition of the mucosal and submucosal tissue layers after the endoscopic procedure.

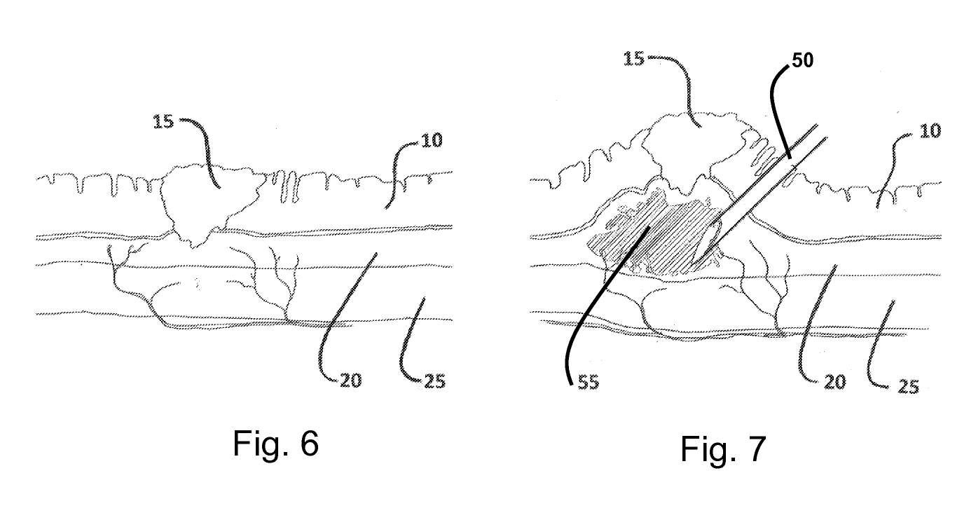

FIGS. 6-7 are schematic cross-sectional views of a portion in the GI tract showing the mucosal tissue layer 10, the submucosal tissue layer 20 and the muscularis propria 25, and illustrate an injection device method for performing a fluid-assisted endoscopic mucosal resection of diseased tissue 15 found in the mucosal tissue layer 10 and a portion of the submucosal tissue layer (see FIG. 6), in accordance with another embodiment of the disclosure.

As shown in FIG. 7, an injection device may include an injection needle 50 that may be inserted into rectum or esophagus by any suitable means, such as through a lumen of an endoscope (not shown), so that the distal end portion of the injection needle may be positioned in the vicinity of a target site. The injection needle 50 may include a hollow lumen through which the injectable material 55 may flow. The distal end of the needle 50 may include a sharp edge configured to pierce tissue, so that the distal end of the needle 50 may be positioned within the submucosal tissue layer 20 in order to deliver a cushion of the injectable material 55 within the submucosal tissue layer 20, lifting the mucosal tissue layer 10. The amount of injectable material to be injected may depend on various factors, such as, for example, type of procedure performed, type of resection instrument used, size of the diseased tissue, or desired degree of cushioning.

Once the injectable material 55 is injected and a stable cushion is provided beneath the diseased tissue 15, a suitable endoscopic resection device having a suitable cutting member (e.g., snare, knife, biopsy forceps, scissors, etc.) may be used to remove the diseased tissue 15, for example, as described above.

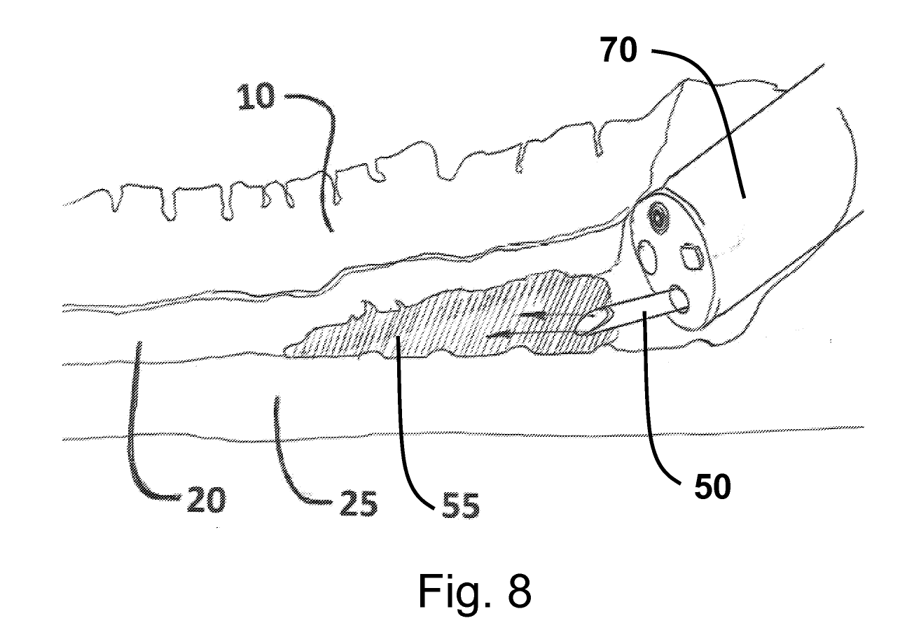

FIG. 8 is a schematic cross-sectional view of a portion of the esophagus showing the mucosal tissue layer 10, the submucosal tissue layer 20 and the muscularis propria 25, and illustrates an injection device method for performing a fluid-assisted POEM procedure, in accordance with an embodiment of the disclosure. In the embodiment shown, an initial incision is made in though the mucosal tissue layer 10 and the submucosal tissue layer 20 (after optionally injecting injectable material between the submucosal tissue layer 20 and the muscularis propria 25), initiating formation of a submucosal tunnel and permitting entry of an endoscope 70 to within the wall of the esophagus, where the muscularis propria 25 is exposed. During the procedure, an injection device which may include an injection needle 50 that may be inserted into esophageal tissue by any suitable means, for example, through a lumen of the endoscope 70, so that the distal end portion of the injection needle may be positioned in the vicinity of a target site. As above, the injection needle 50 may include a hollow lumen through which the injectable material 55 may flow. The distal end of the needle 50 may include a sharp edge configured to pierce tissue, so that the distal end of the needle 50 may be positioned between the submucosal tissue layer 20 and the muscularis propria 25 in order to deliver the injectable material 55 at this location. Upon injection of the injectable material 55, the submucosal tissue layer 20 is dissected from the muscularis propria 25 further exposing the muscularis propria 25 as shown, thereby allowing a health care provider to view and cut circular muscular fibers within the esophagus wall. The amount of injectable material to be injected may depend on various factors, such as, for example, the desired degree of dissection. If further tunneling is desired, the process may be repeated. If desired, dissection may be supplemented using a suitable cutting member, which may be introduced, for example, through a channel of the endoscope 70. If desired, injectable material 55 may be flushed from the site by delivering flushing fluid through a channel of the endoscope 70 (and optionally removing fluid through an endoscope channel). At the conclusion of the procedure, the esophageal incision is closed (e.g., with endoscopic clips or other suitable closure devices).

In another aspect of the disclosure, kits useful in performing a surgical procedure are provided. The kits may include all or a subset of all the components useful for treating a patient.

The kits may include, for example, any combination of two or more of any of the following items: (a) injectable compositions as described herein in a form ready for injection into patient tissue (e.g., provided in one or more pre-loaded syringes), (b) one or more injection needles (e.g., an endoscopic injection needle), (c) one or more tissue resection devices (e.g., snare, knife, scissors), (d) one or more tissue retrieval devices (e.g., net, basket, cap, etc.), (e) one or more combination devices such as devices having tissue injection and tissue resection functions (e.g., a needle combined with a snare), devices having tissue resection and tissue retrieval functions (e.g., a snare combined with a net, basket or cap), or devices having tissue injection, tissue resection, and tissue retrieval functions (e.g., a needle combined with a snare and a net, basket or cap), (f) an endoscope, (g) one or more closure devices (e.g., endoscopic clips), (h) suitable packaging material, and (i) printed material with storage information and/or instructions regarding how to use the items provided within the kit.

### Example 1

Hyaluronic acid sodium salt having a molecular weight ranging from 1×10⁶ Da to 3×10⁶ Da, more preferably from 1.5×10⁶ Da to 2.0×10⁶ Da, for example, Hyaluronic acid sodium salt having a molecular weight of about 1.78×10⁶ Da (available from Sigma) is combined with water to a HA concentration ranging up to 0.15% w/w, for example, ranging from 0.1% w/w to 0.15% w/w, preferably ranging from 0.12% w/w to 0.15% w/w, more preferably, ranging from 0.14% w/w to 0.15% w/w, to form injectable compositions. The injectable compositions are suitable for injection from a syringe (e.g., a standard 10 cc syringe), through an endoscopic needle (e.g., an Interject® sclerotherapy needle from Boston Scientific, Natick, Mass., USA) and into mammalian tissue. When injected into tissue, a bleb would be raised and maintained for adequate duration to be resected. Without wishing to be bound by theory, it is believed that the compositions are successful as they have sufficient concentration to effectively raise a bleb and allow for tissue resection, while not exceeding a concentration limit that would exceed ergonomic force restraints imposed by the delivery means.

### Example 2

Sodium carboxymethyl cellulose (SCMC) having a molecular weight ranging from 0.5×10⁶ Da to 1.0×10⁶ Da, for example, about 0.7×10⁶ Da (available from Acros Organics, Geel, Belgium) is combined with water to a SCMC concentration ranging from 0.1% w/w to 0.25% w/w, to form injectable compositions. The injectable composition s are suitable for injection from a syringe (e.g., a standard 10 cc syringe), through an endoscopic needle (e.g., an Interject® sclerotherapy needle from Boston Scientific, Natick, Mass., USA) and into mammalian tissue. When injected into tissue, a bleb would be raised and maintained for adequate duration to be resected. Without wishing to be bound by theory, it is believed that the compositions are successful as they have sufficient concentration to effectively raise a bleb and allow for tissue resection, while not exceeding a concentration limit that would exceed ergonomic force restraints imposed by the delivery means.

### Example 3

Xanthan gum (available from Sigma) having a molecular weight between 1×10⁶ Da and 50×10⁶ Da is combined with water in a gum concentration ranging from 0.1% to 0.8% w/w, preferably, 0.2% to 0.4% w/w, to form injectable compositions. The injectable compositions are suitable for injection from a syringe (e.g., a standard 10 cc syringe), through an endoscopic needle (e.g., an Interject® sclerotherapy needle from Boston Scientific, Natick, Mass., USA) and into mammalian tissue. When injected into tissue a bleb would be raised and maintained for adequate duration to be resected. Without wishing to be bound by theory, it is believed that the compositions are successful as they have sufficient concentration to effectively raise a bleb and allow for tissue resection, while not exceeding a concentration limit that would exceed ergonomic force restraints imposed by the delivery means.

Although various embodiments are specifically illustrated and described herein, it will be appreciated that modifications and variations of the present disclosure are covered by the above teachings and are within the purview of the appended claims without departing from the spirit and intended scope of the invention.

What is claimed is:

1. An injectable composition comprising water and a polysaccharide, wherein said composition has a viscosity ranging from 100 to 5000 cps and wherein said composition is a sterile composition.

2. The injectable composition of claim 1, comprising sodium in a concentration of concentration of 150 mmol/l or less.

3. The injectable composition of claim 1, comprising sodium in a concentration ranging from 100 mmol/l to 120 mmol/1.

4. The injectable composition of claim 1, wherein the osmolarity of the composition is 310 milliosmoles/liter or less.

5. The injectable composition of claim 1, wherein the polysaccharide is present in the composition in an amount ranging from 0.1% w/w to 0.5% w/w.

6. The injectable composition of claim 1, wherein the polysaccharide is hyaluronic acid sodium salt and wherein the hyaluronic acid sodium salt ranges from 100 to 5000 cps in viscosity.

7. The injectable composition of claim 1, wherein the polysaccharide is hyaluronic acid sodium salt and wherein the hyaluronic acid sodium salt ranges from 0.1% w/w to 0.15% w/w in concentration.

8. The injectable composition of claim 1, wherein the polysaccharide is hyaluronic acid sodium salt and wherein the hyaluronic acid sodium salt ranges from 1×10⁶ Da to 3×10⁶ Da in molecular weight.

9. The injectable composition of claim 1, wherein the polysaccharide is sodium carboxymethyl cellulose and wherein the sodium carboxymethyl cellulose ranges from 100 to 5000 cps in viscosity.

10. The injectable composition of claim 1, wherein the polysaccharide is sodium carboxymethyl cellulose and wherein the sodium carboxymethyl cellulose ranges from 0.1% w/w to 0.25% w/w in concentration.

11. The injectable composition of claim 1, wherein the polysaccharide is sodium carboxymethyl cellulose and wherein the sodium carboxymethyl cellulose ranges from 0.5×10⁶ Da to 1.0×10⁶ Da in molecular weight.

12. The injectable composition of claim 1, wherein the polysaccharide is xanthan gum and wherein the xanthan gum ranges from 100 to 5000 cps in viscosity.

13. The injectable composition of claim 1, wherein the polysaccharide is xanthan gum and wherein the xanthan gum ranges from 0.2% w/w to 0.4% w/w in concentration.

14. The injectable composition of claim 1, further comprising a dye.

15. A syringe filled with the injectable composition of claim 1.

16. A kit comprising the syringe of claim 15 and one or more items selected from (a) an endoscopic injection needle, (b) tissue resection device, (c) a tissue retrieval device, and (d) an endoscope, within a suitable packaging material.

17. A method comprising injection of an injectable composition to a subject, wherein the injectable composition comprises water and a polysaccharide, wherein said composition has a viscosity ranging from 100 to 5000 cps, and wherein said composition is a sterile composition.

18. The method of claim 17, wherein the injectable composition is injected at a target site between a mucosal tissue layer and a submucosal tissue layer in the gastrointestinal tract, or within a submucosal tissue layer in the gastrointestinal tract, so that a surface of the mucosal tissue layer protrudes into the tract; and performing a medical procedure on the protruded surface of the mucosal tissue layer.

19. The method of claim 17, comprising removing tissue raised by the injectable composition.

20. The method of claim 17, wherein the injectable composition is injected at a target site between a submucosal tissue layer and a muscularis propria of an esophagus, so that a tunnel is formed between the submucosal tissue layer and the muscularis propria; and performing a medical procedure on the muscularis propria.
